(19) United States
(12) Patent Application Publication
(10) Pub. No.: US 2007/0259061 A1
(43) Pub. Date: Nov. 8, 2007

(54) Seeds of the genus lecythis as nutritional selenium carrier

(76) Inventor: Winfried Behr, Bonn (DE)

Correspondence Address: JACOBSON HOLMAN PLLC, 400 SEVENTH STREET N.W., SUITE 600, WASHINGTON, DC, 20004, US

(21) Appl. No.: 11/797,353

(22) Filed: May 2, 2007

Related U.S. Application Data
(63) Continuation of application No. 10/969,207, filed on Oct. 21, 2004, now abandoned.
(60) Provisional application No. 60/512,766, filed on Oct. 21, 2003.

(51) Int. Cl. A61K 8/97; A61P 3/02

(52) U.S. Cl. 424/776

(57) ABSTRACT

Subject of the invention is a process of manufacturing a selenium containing composition from selenium containing plant seeds comprising the step of
extracting seeds of selenium containing plants with an apolar organic solvent yielding an extract wherein the selenium containing plants are selected from the group consisting of Lecythis ollaria Loeffling, Lecythis minor Jacquin, Lecythis usitata Miers, and Lecythis tuyrana Pittier, collecting the extract and substantially removing the apolar organic solvent yielding an oily phase having an insoluble residue, isolating the oily phase and the insoluble residue. Subject to the invention are further a method of cold pressing selenium containing plant seeds, selenium containing compositions and nutritional supplements.

### BACKGROUND OF THE INVENTION

Selenium is an essential trace element for human (and animal) nutrition. Its essentiality is due to the fact that it is an integral part of various enzymes (glutathione peroxidases, thioredoxine reductase). In total 35 selenium containing proteins have been determined by labelling experiments with ⁷⁵Se Selenite. Dietary selenium has a powerful antioxidative and immune modulating effect: “Selenium modulates immunity: Se deficiency impairs immunity, Se intakes above those habitually consumed in many Western countries boost immunity and high Se intakes lead to toxic effects and suppression of immunity” (McKENZIE, 2002).

It is known that dietary Selenium deficiency leads to a higher risk for cardiac disease (KIEM, 1984) and cancer (SHAMBERGER, 1971; SCHRAUZER, 1977; HELZLSOUER, 1989; BURNEY, 1989). Supplementation with 200 mcg selenium per day and person reduces cancer incidence by roughly 50% (CLARK, 1996). Selenium plays a role in the detoxification and excretion of the heavy metals Cadmium and Mercury (PARIZEK, 1971, GASIEWICZ, 1976), and probably in the cartilage formation (MÜLLER, P., 1991). Rheumatoid arthritis can be prevented by selenium supplementation (PARNHAM 1995). Acute pancreatitis, an often lethal disease, is partly a consequence of insufficient selenium intake. Medication with selenium compounds improves the prospect of the afflicted dramatically (KUKLINSKI, 1991).

The daily selenium requirement was determined in an equilibrium regression study to be 80 micrograms for (adult) males and 57 micrograms for (adult) females (LEVANDER and MORRIS, 1984).

A national society for nutrition (DEG, 1991) recommends the following amounts as adequate selenium intake:

| Infants | mg/day |
|---|---|
| 0 up to 4 months old | 5-15 |
| 4 up to 12 months | 5-30 |
| 1 up to 4 years | 10-50 |
| 4 up to 7 years | 15-70 |
| 7 up to 10 years | 15-80 |
| Above 10 years | 20-100 |
| Adolescents and adults | 20-100 |

According to various authors the selenium supply by locally produced food is often suboptimal (ROEKENS, 1986; BRÜGGEMANN, 1989). Selenium is predominantly derived from ingested cereals and most cereal provenances have an insufficient selenium concentration (BRÜGGEMANN, 1989).

After the importance of an adequate selenium intake for a balanced nutrition had been understood various products appeared on the market which were intended to secure a sufficient selenium supply. The selenium carrier of these products can be an inorganic selenium salt in the simpler cases. However, inorganic selenium salts are illegal in some countries and in addition the market tends to reject inorganic food additives.

Therefore large quantities of so-called selenium yeast are being produced, that is, a yeast fermented on a substrate containing inorganic selenium salt. During fermentation the inorganic selenium is metabolised into selenium containing amino acids (selenomethionine and selenocysteine) which are then incorporated into the yeast protein (KORHOLA, 1986; HAAS, 1992). Selenium yeast is accepted in many countries as selenium carrier. In others yeast containing products are not popular because of a fear of yeast infections (“The Yeast Connection”). These infections are caused by the genus Candida only and not by the species Saccharomyces cerevisiae which is used for selenium yeast fermentation. Thus there is no scientific basis for the general fear of yeasts, but this aspect has to be taken into consideration when making marketing efforts for selenium containing products.

This dilemma could be avoided by the use of conventional food raw materials which are derived from seleniferous soils and which therefore do contain a certain amount of selenium.

An example of such a selenium rich food is wheat harvested in certain areas of the Midwestern United States of America. Such wheat contains usually only low concentrations of selenium, e.g. 1 to 10 mg Se/kg, in exceptional cases as much as 40 mg/kg (OLSON, 1970). The availability is in addition uncertain and it is expensive as separate harvesting, storage and transport causes additional costs. Wheat plants absorb selenium like most other plants only passively (that is, together with and in substitution of the chemically similar element sulphur) with the consequence that the selenium concentration in the wheat grains remains relatively low.

There are, however, other plants, so-called selenium accumulators, which absorb selenium actively and accumulate (“compartmentalize”) it discriminately in certain plant organs or plant parts. Most known selenium accumulators occur in the seleniferous regions of the USA, they are herbaceous plants, not suitable for human consumption. There exists, however, a well known food provided by a selenium accumulating species: The Brazil nut (Bertholletia excelsa, Humboldt & Bonpland, of the family Lecythidaceae). Bertholletia accumulates selenium in the seeds which were found to contain occasionally as much as 400 mg/kg of selenium in the defatted seed matter (PALMER, 1982). Unfortunately, the selenium concentration in Brazil nuts is very variable, in most cases little or no selenium is present in the seeds. The production of a natural, selenium containing raw material for food supplements is therefore not possible or at least not economical.

Other species of the Lecythidacea family do also accumulate selenium in the seeds. This is known of Lecythis ollaria Loefling, Lecythis minor Jacquin, Lecythis tyurana Pittier and Lecythis usitata Miers (synonym with Lecythis pisonis Cambessèdes).

Lecythis ollaria is described in the handbook “Frutales en Venezuela” of HOYOS (1989). The seeds are oil rich and edible and are used occasionally for oil extraction. The wood of the tree is said to be durable in moist environments and therefore is often used in the construction of dikes and locks.

The species Lecythis minor Jacquin (synonymous: L. elliptica) is described in various books, e.g. “Some Fruits and Nuts for the Tropics” (KENNARD, 1960), “Edible Nuts of the World” (MENNINGER, 1977) und “Tropische Nutzpflanzen” (BRÜCHER, 1977).

Lecythis tuyrana Pittier and Lecythis pisonis Cambessèdes are among the species described in Flora Neotropica (MORI, 1990)

KERDEL-VEGAS (1966) and DICKSON (1969) published on the occurrence of selenium in the seeds of L. ollaria and L. minor. The authors report cases of selenium poisoning caused by consumption of the seeds. Yet both authors stress the variability of the selenium concentration of the seeds, high concentrations were found in the seeds of some trees while the seeds of others growing in close distance did practically contain no selenium.

A high selenium concentration in seeds of Lecythis usitata Miers (synonym with Lecythis pisonis Cambessèdes) was found by ANDRADE (1999).

### DESCRIPTION OF THE INVENTION

The problem of the invention is solved by processes, selenium containing compositions, nutritional supplements and uses of claims 1 to 13.

An object of the invention was to provide a process manufacturing a selenium containing composition and products derived thereof. The invention is based on an unexpected result of experiments which determined the selenium concentration in defatted seeds of Lecythis tyrana Pittier to be 800 mg Se/kg. Investigation further revealed surprisingly that the selenium concentration in the seeds of Lecythis was not only high in individual seeds but also in ton quantity lots collected in large areas. This was unexpected in view of the low average selenium concentration in Brazil nuts.

The process of the invention of manufacturing a selenium containing composition from selenium containing plant seeds comprises the step of
extracting seeds of selenium containing plants with an apolar organic solvent yielding an extract wherein the selenium containing plants are selected from the group consisting of Lecythis ollaria Loeffling, Lecythis minor Jacquin, Lecythis usitata Miers, (synonymous with Lecythis pisonis Cambessèdes) and Lecythis tuyrana Pittier,
collecting the extract and substantially removing the apolar organic solvent yielding an oily phase having an insoluble residue,
isolating the oily phase and the insoluble residue.

As an alternative it is also possible to cold pressing seeds of selenium containing plants selected from the group consisting of Lecythis ollaria Loeffling, Lecythis minor Jacquin, Lecythis usitata Miers (synonymous with Lecythis pisonis Cambessèdes), and Lecythis tuyrana Pittier, and collecting oil pressed out of the seeds.

In the process of the invention regarding the extraction with an apolar organic solvent, the apolar solvent is selected from the group consisting of ethers, preferably petrol ether, esters, preferably ethyl acetate, hypercritical carbon dioxide, alcohols, preferably methanol, ethanol and blends of the above, preferably of ethyl acetate, methanol and ethanol. In a preferred embodiment, the apolar solvents, especially ethyl acetate, methanol and ethanol, may be mixed with each other and with up to 40% of water.

The use of mixtures of ethanol or methanol with water, preferably at a ratio of 70:30 (v/v) is especially advantageous, because aflatoxin, resulting from infection of the seeds with Aspergillus flavus, is removed by those solvents.

Such toxins are often found as a result of lack of experience of the nut collectors. Though the treatment with such solvents was known previously for the removal of aflatoxin, it was found that surprisingly it also is efficient in the extraction of the invention. An extraction with a mixture of ethanol or methanol with water may also be useful in addition to an extraction of the invention.

The process of the invention opens the way to an economical production of raw materials with a high natural selenium concentration to be used in the preparation of selenium containing food supplements or of cosmetic products.

It was a further surprise that most of the selenium of Lecythis seeds remains in the press cake while only little ends up in the oil. Press cake of Lecythis seeds contains, depending on provenance, up to more than 5000 mg Se/kg press cake while the oil usually has a selenium concentration of only around 4 mg/kg. Both components can be used for the manufacture of food supplements or cosmetics, directly or after adjustment of the selenium concentration to a desired level by blending with a carrier.

According to the invention, a process of manufacturing selenium containing composition from selenium containing plant seeds may comprise a process of claim 3 followed by treating the obtained press cake in a process of claim 1. In such a combined process, the press cake obtained after pressing the oil mechanically out of the seeds is subjected to an extraction with an apolar solvent.

The invention is further described by the following non limiting examples.

### EXAMPLES

### Example 1

### Seeds of Lecythis ollaria Loefling

A specimen of a seed of Lecythis ollaria is shelled:

Weight of the shell: 1.3 g

Weight of the seed kernel: 1.1 g

The seed is cut into thin chips which are extracted four times with 10 g petrol ether each time. The mixture is magnetically agitated by a magnetic agitator for 8 minutes each time. After sedimentation the supernatant solution is decanted and evaporated. An oily extract remains. The insoluble portion disintegrates during agitation into a powder. The powder is filtered off an dried.

Oily extract: 0.7 g

Residue: 0.3 g

Loss: 0.1 g

The residue is analysed for selenium. Result: 2200 mg Se/kg.

### Example 2

### Seeds and leaves of Lecythis ollaria Loefling

The origin of the seed of example 1 is exactly known. It was picked from a solitary L. ollaria tree growing on a river bank. One year later a seed of the same tree is analysed again. This time the selenium value of the defatted seed is 1300 mg Se/g. An air dry leaf of the tree contained 0.27 mg/kg selenium. Thus, the accumulation of selenium takes place in the seeds only.

### Example 3

### Seeds of Lecythis ollaria Loefling

The example 1 is repeated with a further seed specimen of Lecythis ollaria. The seed meal was found to contain 1500 mg selenium/kg.

### Example 4

### Seeds of Lecythis tuyrana Pittier

The examples 1 and 2 are repeated with seeds of Lecythis tuyrana.

Selenium content of the defatted seed matter: 800 mg Se/kg

### Example 5

### Seeds of Lecythis minor Jacquin

The examples 1 and 2 are repeated with seeds of Lecythis minor.

Selenium content of the defatted seed matter: 14000 mg Se/kg.

### Examples 6 to 9

### Seeds of Lecythis minor Jacquin

The examples 1 and 2 are repeated with additional seeds and corresponding soil samples of various specimens of Lecythis minor. The results are combined in table 1.

### Example 10

A larger quantity of seeds of Lecythis minor are cold pressed. The selenium content of the press cake is found to be 5500 mg Se/kg

A sample of the extracted oil after filtration was found to contain 4.9 mg Se/kg

### Example 12

5700 kg of seeds of Lecythis minor are cold pressed. 1840 kg of seed meal and 3393 kg seed oil is recovered.

The selenium content of the press cake is found to be 4450 Se/kg

### Example 13

8500 kg of seeds of Lecythis minor are cold pressed. 3187 kg press cake and 5152 kg oil are recovered.

The selenium content of the press cake is found to be 5850 Se/kg

### Example 14

4850 kg of seeds of Lecythis minor are cold pressed. 1222 kg press cake and 3588 kg oil are recovered. The press cake is extracted with ethanol. The final yield of dry seed meal is 870 kg.

The selenium content of the dry seed meal is found to be 5475 Se/kg

TABLE 1
Selenium concentration of defatted seed meal of Lecythis ollaria and
Lecythis minor.

| | | Selenium content in seed meal |
|---|---|---|
| Example | Species | mg/kg |
| 1 | L. ollaria | 2200 |
| 2 | L. ollaria | 1300 |
| 3 | L. ollaria | 1500 |
| 4 | L. tyurana | 800 |
| 5 | L. minor | 14000 |
| 6 | L. minor | 11000 |
| 7 | L. minor | 8800 |
| 8 | L. minor | 3200 |
| 9 | L. minor | 2100 |

TABLE 2
Selenium concentration of crude and defatted seed meal of production
nlots of Lecythis minor.

| | Amount | | Se Concentration |
|---|---|---|---|
| Experiment | of processed seeds | Seed meal yield | in the seed meal |
| 12 | 5700 kg | 1840 kg | 4450 mg Se/kg |
| | | (cold pressed, not | |
| | | extracted) | |
| 13 | 8500 kg | 3187 kg | 5850 mg Se/kg |
| | | (cold pressed, not | |
| | | extracted) | |
| 14 | 4850 kg | 870 kg | 5475 mg Se/kg |
| | | (cold pressed, not | |
| | | extracted) | |

### REFERENCES

ANDRADE, MAIA, J., STREICH, R., 1999: Seed composition of Amazonian Lecythidaceae species. J. Food Compositon and Analysis, vol 12, p 37-51, 1999

BURNEY, P. G. et al., 1989: Serologic precursors of cancer: serum micronutrients and the subsequent risk of pancreatic cancer. Am-J-Clin-Nutr. 1989 May; 49(5): 895-900

BRÜCHER, H., 1977: Tropische Nutzpflanzen, Springer Verlag, 1977, S. 410

BRÜGGEMANN, J. et al., 1989: 40. Tagung für Getreidechemie in Detmold, 8.-9.6.1989

CLARK, L. C., 1996: Effects of Selenium Supplementation for Cancer Prevention in Patients With Carcinoma of the Skin. JAMA, Dec. 25, 1996, vol 276, No. 24, 1957-1963

DEG, 1991: Deutsche Gesellschaft für Ernährung. Empfehlungen für die Nährstoffzufuhr. S. 75

DICKSON, J. D., 1969): Notes on Hair and Nail Loss After Ingesting Sapucaia Nuts (Lecythis elliptica). Econ. Bot., 23, 133-134

GASIEWICZ, T. A.; SMITH J. C., 1976: Interactions of cadmium and selenium in rat plasma in vivo and in vitro. Biochim. Biophys. Acta, 428, 113, 1976

HAAS, H. J. et al.: 1992: Selenoproteins in Mitochondria and Cytosol of Saccharomyces uvarum. J. Trace Elem. Electrolytes Health Dis. Vol 6, 1992, pp. 71-74

HELZLSOUER, K. J. et al., 1989: Selenium, lycopene, alpha-tocopherol, beta-carotene, retinol, and subsequent bladder cancer. Cancer Res. 1989 Nov 1; 49(21): 6144-8

HOYOS, J. F., 1989: Frutales en Venezuela, Soc. de Cienc. Nat. La Salle, Monografia No. 36, Caracas, 1989, S. 124

KENNARD; WINTERS, 1960: Some Fruits and Nuts for the Tropics, p.78, Miscellaneous Publication No. 801, US Dept. of Agriculture

KERDEL-VEGAS, F. (1966): Econ. Bot., 20(1966) 187-195.

KIEM, J.; FEINENDEGEN, L. E., 1984

Platelets and Thrombosis: Selenium and other Mineral Elements Trace Element—Analytical Chem. in Medic. and Biol. Vol 3, p. 339

KORHOLA, M. et al., 1986: Selenium Yeast. Annals of Clinical Research 18, 1986, 65-68

KUKLINSKI, B.; BUCHNER, M., 1991: Akute Pankreatitis—eine “free radical desease”. Letalitätssenkung durch Natriumselenittherapie. Z. gesamte Inn. Med. 46 (1991) 5, 145-49

LEVANDER O. E.; MORRIS, V. C., 1984: Dietary selenium levels needed to maintain balance in North American adults consuming self-selected diets. The American Journal of Clinical Nutrition 39: May 1984, pp 809-815

McKENZIE, R. C., 2002: Selenium and the Immune System, p. 229 in “Nutrition and Immune Function” Ed. P. C. Calder, C. J. Field and H. S. Gill, CABI Publishing, ISBN 0-85199-583-7

MENNINGER, A. E., 1977: Edible Nuts of the World. Horticultural Books, Inc. Stuart. Florida 33494, p. 37

MORI, S. A.; PRANCE, G. T., 1990: Flora Neotropica, Monograph 21 (II), Lecythidaceae-Part II, Published by The New York Botanical Garden, p. 315 MÜLLER, P., 1991: Materie und Prozesse, Tagungsbericht der Deutschen Gesellschaft für Naturforscher und Ärzte, WVA, Stuttgart, S. 251 (W. Gerok ed.)

OLSON, O. E. et al., 1970: Investigation on Selenium in Wheat. Phytochemistry, 1970, Vol 9, pp. 1181 to 1188

PALMER, I. S. et al., 1982: Toxicity of Selenium in Brazil Nuts to Rats. Journal of Food Science, vol. 47, 1595-1597

PARIZEK J. et al., 1971: The detoxifying effects of Selenium, in: Mertz, W. & Cornatzer, W. E., ed., Newer trace elements in nutrition New York, Marcel Dekker, Inc., p. 85-122

ROEKENS E. J. et al., 1986: Dietary selenium intake in Belgium for different population groups. Z. Lebensm Unters Forsch (1986) 182:8-13

SCHRAUZER G. N., u.a., 1977: Cancer mortality correlation studies, III Statist. association with dietary Selenium intakes, Bioinorg. Chem. 7, 23, 1977

SHAMBERGER, R. J; TYTKO, S., Willis C., 1971: Selenium distribution of human cancer mortality, CRC Crit. Rev.: Clin Lab. Sci. 2, 211, 1971

1-13. (canceled)

14. A process of manufacturing a selenium-containing composition from selenium-containing plant seeds comprising cold pressing selenium-containing seeds selected from the group consisting of Lecythis minor Jacquin, Lecythis ollaria Loeffling, and Lecythis tuyrana Pittier, to obtain oil pressed out of the seeds and a press cake, wherein the press cake is used in the preparation of selenium-containing food supplements or selenium-containing cosmetic products.

15. A process of manufacturing a selenium-containing composition from selenium-containing plant seeds comprising the steps of
cold pressing selenium-containing seeds selected from the group consisting of Lecythis minor Jacquin, Lecythis ollaria Loeffling, and Lecythis tuyrana Pittier, to obtain oil pressed out of the seeds and a press cake, followed by
extracting the press cake with an apolar organic solvent, yielding an extracted press cake.

16. The process according to claim 15 wherein the apolar organic solvent is selected from the group consisting of an ether, an ester, an alcohol, petrol-ether, ethyl acetate, hypercritical carbon dioxide, and blends of ethyl acetate.

17. The process according to claim 15 wherein the press cake is used in the preparation of selenium-containing food supplements or selenium-containing cosmetic products.
